(19) United States
(12) Patent Application Publication
(10) Pub. No.: US 2024/0315937 A1
(43) Pub. Date: Sep. 26, 2024

(54) STABLE EFFICIENT COSMETIC PREPARATIONS

(71) Applicant: YISSUM RESEARCH DEVELOPMENT COMPANY OF THE HEBREW UNIVERSITY OF JERUSALEM LTD., Jerusalem (IL)

(72) Inventors: Simon BENITA, Tel Aviv (IL); Taher NASSAR, Kfar Tur'an (IL)

(21) Appl. No.: 18/259,836

(22) PCT Filed: Jan. 11, 2023
(86) PCT No.: PCT/IL2022/050035

§ 371 Date: Jun. 29, 2023

Related U.S. Application Data
(60) Provisional application No. 63/136,192, filed on Jan. 11, 2021.

(51) Int. Cl. A61K 8/34; A61Q 17/04; A61Q 19/08

(52) U.S. Cl. CPC A61K 8/347; A61Q 17/04; A61Q 19/08; A61K 2800/30; A61K 2800/522

(57) ABSTRACT

The invention generally concerns formulations of cannabinoids for cosmetic purposes. To that end, the invention provides formulations with improved stability and topical delivery, as well as improved efficacy in terms of anti-inflammatory, anti-aging effects, and skin protective effects overall.

### TECHNOLOGICAL FIELD

The invention generally relates to the field of cosmetic formulations, and specifically to formulations containing single or mixtures of cannabinoids or extracts of cannabis. To that end, the invention provides formulations with improved stability and topical delivery, as well as improved efficacy in terms of anti-inflammatory, anti-aging effects, and skin protective effects overall.

### BACKGROUND

The cosmetic industry is constantly seeking new and pioneering products that combine both-a desired cosmetic effect and an efficient topical delivery. Despite numerous advances in the development of innovative materials and delivery methods, most of the cosmetic products seen on the market still use conventional formulations. Cosmetic products usually have a complex composition of materials with different physicochemical properties serving different purposes and activities. The art of formulating, in general, and in cosmetics in particular, is to try and achieve a single carefully balanced physical entity without losing the specific purposes and activities of the individual materials.

One approach to achieve a successful formulation would be a modification of physicochemical properties of a specific material by salt formation or a reduction of particle size, for example. Such approaches, however, are not free of limitations. Formation of salts is inapplicable for neutral compounds and is not always commercially feasible for weak acids and bases. In some cases, such salts have tendency to revert to the original acid- or base-form, resulting in the formation of aggregates during the process of making the formulation or upon its application onto the skin. A reduction particle size has its own limitations, with known examples of handling difficulties and poor wettability of very fine powders. These problems have been partially overcome by more recent formulation strategies using new types of surfactants, permeation enhancers, solid dispersions, and nanoparticles. Notwithstanding, formulating lipophilic and combinations of lipophilic and hydrophilic actives, the combinations which are characteristic of cosmetic products, still imposes significant challenges.

Recent evidence suggests that cannabinoids, particularly cannabidiol (CBD), provide interesting benefits for the skin. As a result, cannabinoids have recently emerged as a new trend in cosmetics, with the subsequent attempts to try and produce successful cannabinoid formulations to satisfy the increasing demand for cannabinoid-based cosmetic products. The use of cannabinoids in cosmetics, however, remains a subject to controversy due to legal and regulatory issues associated with obtaining cannabinoid raw material, its manufacturing and commercialization.

Cannabinoids can be obtained from natural sources from three main types of a cannabis plant, Cannabis sativa, Cannabis indica, and Cannabis ruderalis, or synthesized de novo (synthetic), or by modification of natural cannabinoids (semi-synthetic). More than 200 natural cannabinoids were identified so far, and classified into 12 main classes: tetrahydrocannabinol (THC), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM) types, including extended series of derivatives and precursors.

Cannabinoids act on the endogenous endocannabinoid system (ECS). In the skin, ECS participates in a number of processes underlying cell differentiation, growth and survival, inflammatory and immune responses, nociception, and hair growth. Dysregulations of ECS have been associated with various skin conditions and disorders. ECS basically operates via two G protein-coupled receptors: cannabinoid type receptors 1 and 2 (CB1 and CB2), both are expressed in various types of cutaneous cells such as hair follicles, sensory neurons, immune cells, sebaceous glands, and keratinocytes. CB1 have been mostly implicated in the regulation of pain and excessive neural activity in the skin, and more recently with studies of keratinocytes and its effect on the production of pro-inflammatory cytokines, in the regulation of inflammatory responses and the protection of skin barrier. The anti-inflammatory function of CB2 in the skin is more established, phenotypically and on the molecular level. It is thought to act primarily via inhibition of macrophage M1/M2 polarization and down regulation of pro-inflammatory cytokines.

Cannabinoids are exogenous ligands of the endocannabinoid CB1/2 receptors. Some cannabinoids have been related to specific effects on the skin such as antioxidant, antimicrobial and anti-inflammatory activities, and more recently, photoprotection.

A specific example is cannabidiol (CBD), the second most abundant cannabinoid in Cannabis sativa. Unlike THC, it is not psychotropic. It is gaining an increasing interest as a single cannabinoid, in purified or synthetic forms, in the design of nutraceutical and various medicinal products. In cosmetics, CBD has been related to a number of valuable properties such as antioxidant, moisturizing, anti-inflammatory, analgesic and wrinkle reducing effects, and skin anti-aging effects overall.

The problem remains, however, is that cannabinoids are highly lipophilic which makes them practically insoluble in water. CBD, for example, has a calculated log P=6.33, meaning a high tendency to remain in a lipid phase instead of an aqueous phase. Therefore, side by side with the growing interests in CBD in cosmetics, there are increasing attempts to achieve successful CBD formulations and its effective delivery to the epidermal layer of the skin. In addition, cannabinoids and THC and CBD in particular are highly unstable at room temperature and sensitive to oxidation and light. All these properties make the task of formulating cannabinoids highly challenging, moreover achieving formulations with a prolonged shelf-life and stable characteristics under commercial storage conditions required from cosmetic products.

### General Description

The main objective of the present invention has been to design and develop effective and safe formulations of cannabinoids for cosmetic applications. Cosmetic products, being the most widely used topical products, are subject to rigorous regulation criteria of safety and long-term stability. Apart from the conventional toxicology and microbiology testing, cosmetic products are further required to meet strict standards of selective topical absorption to exclude potential systemic exposure to various actives.

On the other hand, the highly competitive nature of the cosmetic market and the constant demand for newer products, drive the cosmetics industry and R&D towards discovery and design of new more potent actives with improved cosmetic efficacy and commercialization potential. As a result, there is a constant need to find new formulations of the new actives, addressing the emerging problems of lack of solubility, penetration, and/or physicochemical instability.

In other words, the issues of efficacy, targeted delivery and safety and the careful balance of benefits therefrom, which apply to all regulated foods and drugs, are even more applicable to cosmetic products.

Cannabinoids represent a family of actives that is relatively new in the fields of dermatology and cosmetics, where they are gaining an increasing interest due to the growing evidence of their ability to treat or mitigate many inflammatory skin conditions and even skin cancer. Today, with a better understanding of the ECS systems, we know that the human skin expresses the two essential ECS components, the CB1/2 receptors, which paves the way for the use of cannabinoids as external ligands.

As for the use of cannabinoids in cosmetics, an increasing number of recent studies reinforces the notion of their potential benefits for topical applications. While the immune-regulatory effects of orally administered cannabinoids are well documented, there is now emerging evidence of analogous effects of topically administered cannabinoids on skin conditions and sensitive skin. Specific examples are the improving effect of CBD, CBC, THCV and CBDV on acne-prone skin, of CBG and CBGV on the regulation of sebum production in dry skin, some preliminary findings on CBN and CBD as potential sunscreens. In addition, almost all cannabinoids have antibacterial and antioxidant actions. Apart from CBD, however, there is an apparent lack of safety studies.

CBD in particular has been implicated in regulation of cell cycle, functionality of the immune cells and suppression of a wide range of pro-inflammatory cytokines. In the context of skin, CBD was shown to act as a transcriptional repressor controlling the proliferation and differentiation of skin cells. It was shown to induce nuclear export and degradation of BACH1, a transcription factor regulating ROS production, and thereby to reduce oxidative stress and skin aging. More recently, it was shown to enhance the activity of antioxidant enzymes such as SOD and TrxR in UV irradiated keratinocytes, and to inhibit lipid peroxidation. And in a rat model, topical application of CBD onto the skin exposed to UV radiation has led to normalization of the expression of keratinocyte proteins, on the levels of biosynthesis and degradation.

Therefore, owing to its wide range of activities and specifically its antioxidant and anti-inflammatory properties, CBD is considered one of the most promising candidates for the design of new cosmetic formulations and new protective treatments for the skin.

Natural and more complex cannabinoid mixtures such as extracts or oils of cannabis and hemp hold additional advantages due their content of flavonoids, terpenes, carotenoids and phytosterols which contribute to the anti-inflammatory and anti-aging actions. They further contain tocopherol and chlorophyll which are known antioxidants. In addition, their content and ratio of the two essential fatty acids, ω-6/ω-3, makes them ideal for the skin due to rapid absorption. Therefore, these sources of cannabinoids represent yet another line of candidates for designing cosmetic formulations with broad applications to all skin types and specific applications to sensitive skin.

The strength of the present technology is in providing an effective and operative solution to the main problems associated with the use of cannabinoids in cosmetics: (1) finding formulations with attractive properties of homogeneity, solubility and texture; (2) formulations with a prolonged stability and shelf life in terms of preservation actives and other core properties; and further, (3) formulations having an effective and targeted delivery profile which is restricted to the epidermal layer of the skin.

Using the example of CBD, the inventors have succeeded to achieve smooth, non-greasy and water-based cannabinoid formulations with a complete dissolution of the cannabinoid active up to the final concentration of 2% (w/w). Importantly, these features were reproducible and achieved in several variant formulations (see EXAMPLES 1-5).

Further, these features were achieved without adding to significant amounts of ethanol or other monohydric alcohols to the formulations. In fact, the most successful formulations included only up to 1% ethanol or no ethanol at all (EXAMPLE 1). Ethanol is a known additive in cannabinoid formulations to facilitate solubility and permeability of cannabinoids through the skin. Ethanol, however, and other simple alcohols, is harmful to the sebum layer and due its hygroscopic properties can cause skin dehydration, and in cases of sensitive and eczema-prone skin can lead to additional damages. Therefore, achieving a successful formulation of cannabinoids with a minimal presence of ethanol or no ethanol at all is highly unexpected and advantageous.

Surprisingly, the advantageous features of homogeneity, color, texture, and a complete dissolution of the cannabinoid actives that are characteristic of the present formulations could be attributed to the specific content of chelators and antioxidants, and specifically a combination of ethylenediaminetetraacetic acid (EDTA) and synthetic antioxidants such as butylated hydroxytoluene (BHT), butylated hydroxyanisole (BHA) and/or tert-butyl hydroquinone (TBHQ), and antioxidant oils such as natural or synthetic medium chain triglycerides (MCTs) and/or certain types of vegetable oils comprised in the formulations (EXAMPLE 1).

Still further, the features of homogeneity, color and texture remained stable at room temperature for a period of at least 6-9 months, and for up to 12 months as regards the preservation of actives and other parameters (EXAMPLE 2). In addition, when tested in the antimicrobial preservation challenge test across the entire range of relevant microorganisms, the cannabinoid formulations were found to meet all the requirements of Pharmacopoeia (EXAMPLE 3). Importantly, the present experiments yielded a set of concrete guidelines as to the type of a storage container and a storage temperature that can further enhance the stability of the present cannabinoid formulations.

In terms of targeted delivery to the skin, the present cannabinoid formulations were demonstrated to have a strictly targeted epidermal distribution. When tested by the static diffusion method (Franz cells) in various layers of the skin, the formulations were more efficient by about 3-fold than the respective steroid control in enhancing the penetration of the cannabinoid active primarily into the epidermal layer (EXAMPLE 4). No active was observed in the receptor compartment, thus further supporting the notion of a localized and restricted permeability of cannabinoids. These findings were reproduced in a consecutive independent experiment (EXAMPLE 5).

In terms of anti-inflammatory effects, when tested in an artificially induced inflammation model (LPS) in samples of skin, the present cannabinoid formulations reduced the secretion/expression of the three major pro-inflammatory cytokines in the epidermal tissue, IL-6, IL-8 and IL-1β. Specifically for IL-1β, the reduction has exceeded the respective steroid control (EXAMPLE 5). Interestingly, it was shown that said reduction was further dependent on the type of oil formulated with the cannabinoids, and that cannabinoids formulated with certain types of oils can have additive and synergistic effects on the reduction of pro-inflammatory cytokines in the epidermal tissue.

As to the tested pro-inflammatory cytokines, IL-6, IL-8 and IL-1β were implicated in various inflammatory processes in the skin. Previous studies have shown that the levels of all three cytokines are elevated in UV irradiated human keratinocytes. It has been long established that UV irradiation is a strong factor in skin aging characterized by loss of skin elasticity and certain epidermal components, appearance of fine lines and wrinkles, increased epidermal permeability, and delayed wound healing. Chronic inflammation, in general, have been associated with many age-related disorders such as Alzheimer's, cardiovascular disease, diabetes and cancer, which coined the term “inflammaging”. All three cytokines, IL-6, IL-8 and IL-1β, were implicated in chronic systemic age-related inflammation or inflammaging. In topical disorders, IL-6 was strongly implicated in the pathogenesis of asthma and regulation of wound healing, and IL-8 and IL-1β in the pathogenesis of psoriasis and other inflammatory conditions of the skin.

In other words, IL-6, IL-8 and IL-1β constitute bona fide markers for establishing the effect of candidate formulations on inflammatory skin conditions and skin aging.

Overall, the presently disclosed technology has succeeded to achieve a series of topical formulations with a sufficiently high content of cannabinoids and other components providing them with a number of important skin protective benefits, such as skin moisturizing and soothing, and protecting the skin against photo- and oxidative stress, chronic inflammation, and skin aging. The present formulations are characterized with a distinguished epidermal delivery profile and a proven anti-inflammatory activity, as well as, with a prolonged stability and shelf-life and a sufficient preservation of antimicrobial content. These features make the present formulations superior candidates for the design of products for various medicinal and non-medicinal topical applications. Apart from therapeutics and therapeutic delivery systems, they can be further adapted for a variety of cosmetic applications in the forms of creams, lotions, gels, sprays, cosmetic masks and solid or liquid soaps.

One of the advantageous of the present formulations is in their flexibility and the ability to incorporate additional substances which proven benefits to the skin such as minerals, vitamins, oils, and skin superfoods. Notable examples are vitamins A, B3, B5, C and E, oils of coconut, jojoba, rosehip, almond and olive oil, and complex extracts such as aloe vera, turmeric, certain types of sea moss and fruits. More broadly, the formulations of the invention can incorporate any substance recognized as “GRAS” (Generally Recognized As Safe) by the FDA or EMA (the FDA equivalent in Europe).

Additional, but not mandatory, improvement can be provided by adapting the formulations to include nano-encapsulated cannabinoid forms. Nanoemulsions have become attractive delivery systems for pharmaceutical, food and cosmetics industries due to their ability to maintain a lower content of surfactants, relative stability, lack of toxicity, low viscosity, and translucent appearance, which are critically important for cosmetics. In addition, encapsulation of actives in small particles or droplets provides a closer contact with the skin and a greater exposure to actives, and thus enables to improve the delivery and efficacy of actives onto the skin.

Ultimately, the present technology provides a series of methods, uses and guidelines on how to apply the presently disclosed formulations so as to maximize their therapeutic and cosmetic benefits to the skin.

### BRIEF DESCRIPTION OF DRAWINGS

To better understand the subject matter and to exemplify how it may be carried out in practice, embodiments will now be described by way of non-limiting examples with reference to the following drawings.

FIG. 1 illustrates the feature of advantageous permeability of the cannabinoid creams of the invention with the example of CBD. Figure shows the amount of CBD in different layers of human skin tested in Franz diffusion cell (static diffusion method) after topical administration of the formulation CBD #27 containing 100 μg CBD (2% w/w). CBD amounts were measured in the different skin layers 6 hours after exposure compared to the respective CBD-ethanol control. Values are mean±SD (N=3).

FIGS. 2A-2C illustrate the anti-inflammatory effect of the cannabinoid creams of the invention as revealed the LPS-induced model in vitro by the expression of three main pro-inflammatory cytokines (IL-6, IL-8 and IL-1β). Skin samples were treated topically with the formulation CBD #27 (15 μg/cm²), vehicle (MCT), and clobetasole propionate (2.5 μg/cm²) as a steroid control in the course of 24 h. The levels of IL-6 (2A), IL-8 (2B) and IL-1β (2C) were measured in the medium by ELISA. Values are calculated as means±SD from 4-6 replicates from two independent skin donors and normalized to untreated samples (*p<0.05, ** p<0.01, *** p<0.005. **** p<0.001).

FIGS. 3A-3B illustrate the contributing anti-inflammatory effect of antioxidant oils comprised in the cannabinoid creams of the invention. The experimental conditions were analogous to FIGS. 2A-2C, apart from dexamethasone as a steroid control and IL-1β (3A) and IL-6 (3B) as tested cytokines. Results are normalized to LPS-induced untreated skin (*p<0.05, ** p<0.02).

### DETAILED DESCRIPTION OF EMBODIMENTS

In the broadest sense the invention can be articulated in terms of advantageous water-based formulations containing single or mixtures of cannabinoids or a cannabinoid material that are suitable for medicinal and non-medicinal topical applications.

The term “medicinal topical application” encompasses herein any topical application of the present formulations involving cutaneous (the skin) and/or mucous surfaces (the nose, internal parts of the lips, etc.) that are meant to treat or alleviate symptoms of clinical conditions such as psoriasis, eczema, acne, skin rashes, hives, shingles, contact dermatitis, rosacea, sun burns, and others.

The “non-medicinal topical applications” predominantly refer to cosmetics, implying use of the present formulations for treating, restoring, or improving one's complexion and/or appearance of the skin or hair on any part of the body (face, limbs, head, etc.).

The term “water-based” implies herein predominance of the water component, which can be broadly defined as the water component being present the range between 20-90% of the total weight of the formulation (w/w), and specifically in the ranges between 20-30%, 30-40%, 40-50%, 50-60%, 60-70%, 70-80% and 80-90% of the total weight of the formulation (w/w).

Thus, in numerous embodiments the formulations of the invention can comprise water in the range between about 20% to about 90% of the total weight (w/w).

In further embodiments the formulations can comprise water in the range between about 50% to about 60% of the total weight (w/w).

The terms “cannabinoid” or “cannabinoid material” imply herein a class of chemical compounds, cannabinoid/cannabinoid agonists/cannabinoid-related compounds, capable of binding with various affinities on the endogenous cannabinoid receptors (CB1 and CB2). They encompass herein the endocannabinoids naturally found in humans and animals, the phytocannabinoids (also natural cannabinoids) extracted and/or purified from cannabis and certain types of other plants, the semisynthetic cannabinoids produced by artificial modifications these latter, and the synthetic cannabinoids synthetized de novo by various methods.

In numerous embodiments, these terms refer to single and combinations or mixtures of cannabinoids belonging to one or more of the main cannabinoid classes, namely: tetrahydrocannabinol (THC), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM) classes. The differences between them and their respective structures are shown in Table 1.

TABLE 1
General structure of the main classes of cannabinoids

| Class | Structure |
|---|---|
| Cannabigerol CBG | 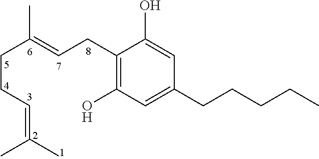 [SMILES: CCCCCc1cc(O)c(C/C=C(\C)CCC=C(C)C)c(O)c1] |
| Cannabichromene CBC | 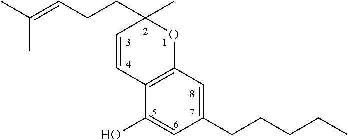 [SMILES: CCCCCc1cc(O)c2c(c1)OC(C)(CCC=C(C)C)C=C2] |
| Cannabidiol-type CBD | 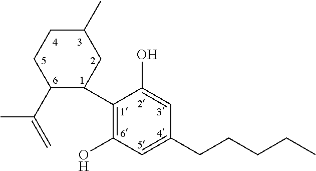 [SMILES: C=C(C)C1CCC(C)CC1c1c(O)cc(CCCCC)cc1O] |
| Tetrahydrocannabinol THC Cannabinol CBN | 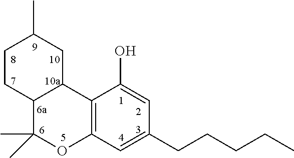 [SMILES: CCCCCc1cc(O)c2c(c1)OC(C)(C)C1CCC(C)CC21] |
| Cannabielsoin CBE | 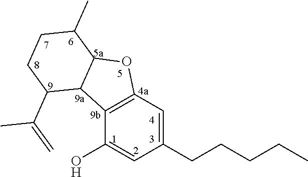 [SMILES: C=C(C)C1CCC(C)C2Oc3cc(CCCCC)cc(O)c3C12] |
| iso-Tetrahydrocannabinole iso-THC | 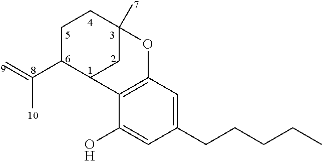 |
| Cannabicyclol CBL | 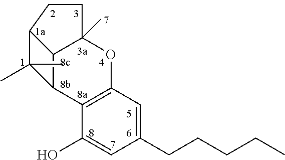 |
| Cannabicitran CBT | 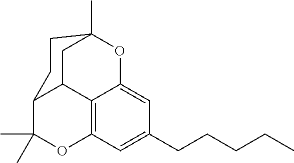 [SMILES: CCCCCc1cc2c3c(c1)OC(C)(C)C1CCC(C)(CC31)O2] |

These terms further encompass herein psychoactive and non-psychoactive compounds selected from one or more of the following: cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C4 (CBD-C4), cannabidivarinic acid (CBDVA), cannabidiorcol (CBD-Ci), delta-9-tetrahydrocannabinolic acid A (THCA-A), del ta-9-tetrahydrocannab inolic acid B (THCA-B), delta-9-tetrahydrocannabinol (THC), delta-9-tetrahydrocannabinolic acid-C4 (THCA-C4), delta-9-tetrahydrocannabinol-CT (THCA-C4), delta-9-tetrahydrocannabivarinic acid (THCVA), delta-9-tetrahydrocannabivarin (THCV), delta-9-tetrahydrocannabiorcolic acid (THCA-Ci), delta-9-tetrahydrocannabiorcol (THC-Ci), delta-7-cis-iso-tetrahydrocannabivarin, delta-8-tetrahydrocannabinolic acid A (D3-THCA), delta-8-tetrahydrocannabinol (DS-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabinolic acid (CBNA), cannabinol (CBN), cannabinol methylether (CBNM), cannabinol, C4 (CBN-C4), cannabivarin (CBV), cannabinol-C2 (CBN-C2), cannabiorcol (CBN-Ci), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinolic (OTHC), delta-9-cis-tetrahydrocannabinol (cis-THC). 3,4,5,6-tetrahtdro-7-hydroxy-a-a-2-trimethyl-9-n-propyl-2,6-methano-2H-I-benzoxocin-5-methanol (OH-iso-HHCV), cannabiripsol (CBR), trihydroxy-delta-9-tetrahydroxycannabinol (triOH-THC), and any other cannabinoid.

They encompass herein cannabinoid precursors, including the main CBG-type precursor and precursors derived therefrom by various cyclization reactions. They refer to cannabinoid acid forms (also 2-COOH or -A forms) which are intermediate precursors of cannabinoids, including the two main cannabinoids precursors THC-A and CBD-A. They further refer to cannabinoid derivatives, synthetic or natural, or synthetically produced from the naturally occurring cannabinoids (semisynthetic).

These terms further encompass single and combinations or mixtures of cannabinoids either purified or extracted from any part of a cannabis variety, C. sativa, C. indica and/or C. ruderalis, or a hemp plant (C. sativa types II, III, IV and V).

Thus, in terms of cannabinoid actives, in numerous embodiments the topical formulations of the invention can comprise a cannabinoid material that comprises any combination of one or more purified natural cannabinoids, synthetic and/or semi-synthetic cannabinoids and/or or extracts of cannabis or hemp plants.

In certain embodiments the formulations can comprise one or more cannabinoids selected from at least one of: THC, CBD, CBN, CBG, CBC, CBL, CBV, THCV, CBDV. CBCV. CBGV, CBGM, or acid precursors or derivatives thereof.

In further embodiments at least one of the comprised cannabinoids can be CBD or a precursor, an acid form, and/or a derivative thereof.

In terms of concentrations, in numerous embodiments the formulations can comprise cannabinoids at a concentration in the range between 0.001-10% of the total weight (w/w), and specifically in the ranges between 0.001-0.005%, 0.005-0.01%, 0.01-0.05%, 0.05-0.1%, 0.1-0.5%, 0.5-1%, 1-5% and 5-10% of the total weight (w/w).

In further embodiments the formulations can comprise cannabinoids at a concentration in the range between 1-2% of the total weight (w/w), and specifically up to about to 1%, 1.1%, 1.2%, 1.3%, 1.4%, 1.5%, 1.6%, 1.7%, 1.8%, 1.9%, 2% of the total weight (w/w) and more.

As has been presently demonstrated, many advantageous features of the present formulations such as long-term stability, homogeneity, a complete dissolution of the cannabinoid actives, and others, derive from the specific combination of their core components. Apart from cannabinoids, the formulations of the invention comprise a combination of chelators and antioxidants.

The terms “chelating agent” or “chelator” broadly refer herein to any chemical compound reacting with metal ions to form stable water-soluble complexes. In many respects chelators can also act as antioxidants, antimicrobial agents and/or preservatives. A notable example of a chelator which is also antimicrobial agent is ethylenediamine-tetraacetic acid (EDTA). EDTA is a synthetic chelator which is widely used in cosmetics and pharma, it can be provided in a form of salt or a complex. Additional examples are natural chelators such as sodium phytate/phytic acid, citric acid/sodium citrate, sodium gluconate.

The term “antioxidant” broadly refers herein to any substance acting as a “free radical scavenger” by preventing or repairing the damages caused by reactive oxygen and nitrogen species (ROS and RNS), including substances acting outside or inside the cells, and substances acting as co-factors in radical detoxifying enzymes (SOD, GPx, and catalase). Notable examples of antioxidants used in cosmetics and pharma are butylated hydroxyanisole (BHA), butylated hydroxytoluene (BHT), propyl gallate (PG) and tert-butyl hydroquinone (TBHQ). Additional examples are hydrophobic and hydrophilic natural antioxidants such as vitamin C, vitamin E, vitamin B3 (niacinamide), vitamin A (retinol), resveratrol, coenzyme Q₁₀, flavonoids, carotenoids, polyphenols and antioxidants present in the Theobroma cocoa butter and minerals such as selenium.

As has been presently demonstrated, a specific combination of chelators and antioxidants, namely EDTA and BHT and potentially also BHA and/or TBHQ, is responsible for a major part of the advantageous features of the present formulations. Apart from this specific combination, the formulations can comprise additional chelators and antioxidants, such as those listed above.

Thus, in numerous embodiments the formulations of the invention can comprise a cannabinoid material and EDTA in a form of salt or a complex and an antioxidant comprising BHT, BHA or TBHQ or a combination thereof.

The term “EDTA salt” refers herein to all known types EDTA salts, and for the most part to disodium EDTA, sodium calcium edetate (sodium calcium EDTA), and tetrasodium EDTA. “EDTA complex” predominantly refers to EDTA metal complexes, including complexes with transition and alkaline metals.

In certain embodiments the antioxidant component can further comprise PG.

In other embodiments the formulations do not comprise PG, as PG is not necessary for the main advantageous features of the present formulations, i.e., the stability, homogeneity, solubility of actives, and others.

In numerous embodiments the formulations can further comprise antioxidant oils.

The term “antioxidant oil” encompasses herein saturated, monounsaturated, and polyunsaturated oils and fats (single and double bonds varieties), with various lengths of backbones and complexities of side chains, in cis and trans orientations, including natural, semisynthetic, and synthetic oils, derivatives and mixtures, which have known antioxidative effects. Notable examples of natural antioxidant oils are lemon oil, rosemary oil, clary sage oil, olive oil, sesame oil and the Theobroma cocoa butter. Additional examples are medium chain triglycerides (MCTs) from synthetic and natural sources. MCTs refer herein to saturated oils and triglycerides with 2-3 fatty acids having an aliphatic tail of 6-12 C-atoms. Examples of natural MCTs are MCTs comprised in coconut oil, palm oil, cocoa butter, and other types of vegetable oils.

Thus, in numerous embodiments the formulations of the invention can comprise at least one natural or synthetic MCT and/or a vegetable oil, or any combination thereof.

In certain embodiments the vegetable oil can be sesame oil.

In numerous embodiments the formulations of the invention can comprise additional types of oils, and especially oils with proven benefits to the skin such as oils extracted from various types of nuts, seeds and plants like avocado oil, canola oil, olive oil, sunflower oil, peanut oil, walnut oil, sunflower seeds oil, flaxseeds or flax oil, corn oil, soybean oil, safflower oil, anise oil, apricot kernel oil, almond oil, cashew oil, rice bran oil, poppy seed oil, cottonseed oil, coconut oil, flaxseed oil, cinnamon oil, clove oil, nutmeg oil, coriander oil, lemon oil, orange oil, palm oil, palm kernel oil, sunflower oil, rapeseed oil, other vegetable oils

One of the distinguishing features of the present formulations is that they are essentially devoid of an alcohol. The term “alcohol” broadly refers to any organic compound that carries at least one hydroxyl functional group (—OH) bound to a saturated C-atom. It predominantly refers to simple monohydric alcohols, including primary (RCH₂OH), secondary (R₂CHOH) and tertiary (R₃COH) alcohols such as methanol, ethanol and isopropyl alcohol and butanol. Methanol is toxic characteristically via ingestion. Ethanol, in contrast, is a known additive in formulations of lipophilic actives. It is further known to facilitate the solubility and permeability of cannabinoids through the skin. On the other hand, ethanol and other alcohols are harmful to the sebum layer, and due to their hygroscopic properties can cause skin dehydration, and in cases of sensitive and eczema-prone skin can lead to additional damages. Thus, formulations essentially devoid of alcohols or with little added alcohols are particularly useful for topical and cosmetic applications.

Thus, in numerous embodiments the formulations of the invention are essentially devoid of monohydric alcohols, and in numerous embodiments it denotes essentially devoid of ethanol. The term “essentially devoid” implies a relatively low content of alcohol or no alcohol at all.

In certain embodiments the formulations can comprise alcohols at a concentration in the range between 0.001-3% of the total weight (w/w), and specifically at a concentration in the ranges between 0.001-0.005%, 0.005-0.01%, 0.01-0.05%, 0.05-0.1%, 0.1-0.5%, 0.5-1%, 1-1.5%, 1.5-2%, 2-2.5%, 2.5-3% of the total weight (w/w).

In other embodiments the formulations can comprise alcohols at a concentration in the range between 0.001-1% of the total weight (w/w), and specifically at a concentration up to about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, and 1% of the total weight (w/w).

In certain embodiments the formulations can comprise no alcohols or the alcohol can be totally absent.

Further, in numerous embodiments the formulations of the invention can comprise additional components from one or more of the following groups: humectants and/or emollients, surfactants and/or emulsifiers, preservatives, antimicrobial agents and/or additional antioxidants.

It should be noted that some of these groups are partially or completely overlapping, meaning specific components can be shared between two or more groups, for example surfactants and emulsifiers, or chelators and antioxidants. In addition, all these components should meet the requirements of FDA approved materials as pharmaceutically compatible or acceptable materials, or cosmetically acceptable materials or materials recognized as GRAS.

The term “humectant” broadly refers herein to any type of a moisturizing agent, or more precisely, an agent absorbing and retaining water or moisture. Notable examples of humectants used in cosmetics and pharma are glycerin, sorbitol (sugar alcohol), hexylene and butylene glycol, urea, and collagen, and polyethylene glycols (PEG) and polypropylene glycols (PPG).

Thus, in numerous embodiments the formulations can comprise as humectants glycerin or glycerin derivatives, propanediol, derivatives of urea, glycols, panthenol, sodium lactate, hyaluronic acid, salicylic acid, alpha-hydroxy acid (AHA) and combinations thereof.

The term “emollient” broadly refers herein to any type of refatting agent or an agent supplying the skin with fat and softening and smoothing the skin, and thereby also reducing evaporation and loss of moisture from the skin. In this respect terms humectants and emollients can be overlapping. Notable examples of natural and synthetic emollients are petrolatum, zinc oxide, paraffin, mineral oil, glycerin, beeswax, olive oil, coconut oil, lanolin, cocoa butter, and such synthetics as butyl stearate and diglycol laurate.

In numerous embodiments the formulations can comprise as emollients triglycerides, free fatty acids, fatty acid esters, silicone-based substances, lanolin, mineral oils, simple or complex esters, or combinations thereof.

In certain embodiments the emollient can be ethylhexanoate and/or dimethicone.

The term “surfactant” (or surface-acting agent) encompasses herein any type of amphiphilic compound containing a hydrophilic and a lipophilic part and tending to concentrate in water-oil interfaces, and thereby functioning as wetting, emulsifying, foaming, thickening, solubilizing, and penetration enhancing agent. This term further encompasses anionic, amphoteric, cationic, nonionic, and silicone-based surfactants. Cationic surfactants have a lot of drawbacks. Notable examples of surfactants used in cosmetics are anionic surfactants used in shampoos or soaps such as sulfosuccinates, alkyl benzene sulfanate, acyl methyl taurates, acyl sarcocinates, the isethionates, propyl peptide condensates, monoglyceride sulfates and fatty glycerol, ether sulfanates; amphoteric surfactants used in soaps such as cocamidopropyl betaine, cocoamphopropionate, and sodium lauraminopropionate; nonionics surfactants used as thickeners and conditioning agents in creams and lotions such as fatty alcohols and fatty alkanolamides, including lauramide diethanolamine (DEA) and cocamide DEA and amine oxides such as lauramine oxide or stearamine oxide. For pharma, surfactants can be selected from polyoxyl castor oil (Cremophor RH40, Kolliphor RH40), polysorbate 80, olcoyl polyoxyl-6 glycerides (Labrafil M1944 CS), polyoxyl 35 hydrogenated castor oil, sucrose distearate, tocopherol polyethylene glycol 1000 succinate (TPGS), lauroyl polyoxyl-32 glycerides (Gelucire), sorbitan monooleate, low-HLB polyoxylglycerides (Labrafil M 1944 CS and Labrafil M 2125 CS), linoleoyl polyoxy-6-glycerides, and combinations thereof.

The term “emulsifier” broadly refers herein to any material slowing the process of separation of a suspension, dispersion, or an emulsion into two or more immiscible phases, and also any substance assisting in the formation and improving the stability of an emulsion, O/W and W/O emulsion. This term further encompasses anionic, cationic, nonionic, liquid crystal, polymeric emulsifiers. Notable examples are anionic emulsifiers such as aloe, sodium PCA, sodium hyaluronate and seaweed extracts; cationic emulsifiers such as quaternium ammonium compounds (distearyldimonium chloride), Quaternium-15 (hexamethylenetetramine chloroallyl chloride) and Quaternium-70 (stearamidopropyl dimethyl myristyl acetate ammonium chloride); nonionic emulsifiers such as polysorbates 80 and 20 (Tweens), salicylic acid, alpha hydroxy acids (AHAs) and zinc oxide; liquid crystal emulsifiers such as lecithin and Olivem (a complex combination of fatty acids); and various polymeric emulsifiers.

Many emulsifiers also act as surfactants. Thus, in certain embodiments the formulations can comprise surfactants and/or emulsifiers which can be cyclopentasiloxane, polysorbate 80 or 20, cetearyl alcohol, glyceryl stearate, cetearyl glucoside or any combination thereof.

Further, the terms “preservative” and “antimicrobial agent” broadly refers herein to any agent, natural or synthetic, with an antimicrobial and/or antioxidant activity. Many preservatives can also act as chelating agents. Notable examples of preservatives used in cosmetics and pharma are parabens (Germaben II, and methyl-, propyl- and butyl-parabens), isothiazolinones (Kathon), phenoxyethanol (Optiphen), organic acids (benzoic acid/sodium benzoate, sorbic acid/potassium sorbate and levulinic and anisic acids).

In numerous embodiments the formulations can comprise one or more natural preservatives and antioxidants selected from benzoic acid, sorbic acid and/or salicylic acid, and combinations thereof.

In other embodiments the formulations can comprise synthetic preservatives and antioxidants selected from parabens, glycol ethers, aldehydes and/or organohalogen and combinations thereof.

In certain embodiments the formulations can comprise preservatives which are phenoxyethanol, caprylyl glycol, chlorphenesin, C12-15 alkyl benzoate, BHT, BHA and combinations thereof.

In numerous embodiments the formulations can comprise additional natural antioxidants selected from: vitamin C, vitamin E, vitamin B3 (niacinamide), vitamin A (retinol), resveratrol, coenzyme Q₁₀, flavonoids, polyphenols or antioxidants comprised in Theobroma Cocoa butter and combinations thereof.

More generally, in numerous embodiments the formulations can comprise various types of minerals, vitamins, oils and/or skin superfoods, and especially those with proven beneficial value to the skin.

In numerous embodiments the formulations can comprise vitamins A, B3, B5, C and E, oils of coconut, jojoba, rosehip, almond and olive oil, and complex extracts such as aloe vera, turmeric, and extracts of sea moss and fruits.

An important advantage of the present formulations resides in their prolonged stability and extended shelf-life at room temperature, or under stress conditions (40° C.) simulating long storage.

In numerous embodiments the formulations can remain stable for at least 6 months and more at room temperature, and specifically, for at least 1, 2, 3, 4, 5, 6, 7, 8, 9, 10, 11, 12 months and 2, 3, 4, 5 years and more at room temperature. The stability can be further improved by specific storage conditions such as specific storage containers, etc.

Yet another important advantage of the present formulations resides in their selective topical delivery of actives and other ingredients to the skin, or in other words, a localized topical delivery of actives and other ingredients with a minimal or almost no systemic exposure. It has been recently demonstrated that this feature can be measured and quantified by the static diffusion method (Franz cells).

Thus, in numerous embodiments the active or the cannabinoid material comprised in the formulations has a preferential distribution to the epidermal layer of the skin. The term “preferential distribution” in the context of skin implies a tendency or a greater propensity of the formulated active to accumulate in certain layers of the skin, and specifically in the epidermal layer which is the target organ. It further implies minimal presence of the formulated active in other layers of the skin and minimal systemic exposure. Preferential distribution in various layers of the skin (e.g., the stratum corneum, epidermis, dermis and residual layer) can be measured by various methods, e.g., static diffusion method (Franz cells) relative to a respective control sample, in this case an alternative formulation or solution of the same cannabinoid (e.g., the cannabinoid in ethanol). Such measurements have been presently demonstrated.

Thus, in certain embodiments this specific feature can be further articulated as the cannabinoid material comprised in the formulations is characterized as having a preferential distribution to the epidermal layer of the skin compared to the other layers of the skin, and specifically the preferential distribution of the cannabinoid material can be increased by at least 2-fold in the epidermal layer than in the other layers of the skin, and more specifically increased by at least 2-, 3-, 4-. 5-, 6-, 7-, 8-, 9-, 10-, 20-, 30-, 40-, 50- and 100-fold and more in the epidermal layer than in the other layers of the skin.

In other embodiments this feature can be further articulated as the cannabinoid material comprised in the formulations is characterized as having a preferential distribution to the epidermal layer of the skin compared to the distribution of the respective cannabinoid control in the epidermal layer, and specifically the preferential distribution of the cannabinoid material to the epidermal layer can be increased by at least 2-fold than the distribution of the respective cannabinoid control in the epidermal layer, and more specifically increased by at least 2-, 3-, 4-, 5-, 6-, 7-, 8-, 9-, 10-, 20-, 30-, 40-, 50- and 100-fold and more in the epidermal layer than the distribution of the respective cannabinoid control in the epidermal layer.

Yet another important advantage resides in their flexibility or adaptability to other formulation technologies such as nanoemulsification and nanoencapsulation methods. It should be noted however that in numerous embodiments the formulations of the invention can be effective per se without additional sophistications and adaptations.

More specifically, for many pharmaceutic and cosmetic applications the cannabinoid material does not necessarily has to be nano-emulsified or encapsulated in micro- or nanoparticles or droplets.

For some applications, however, and especially those requiring an improved topical delivery and permeation or absorption of cannabinoids, the cannabinoid material can be encapsulated in micro- or nanoparticles or droplets, using known in the art technologies such as high-pressure homogenization, micro-fluidization, and ultrasonication, and further, various types of oil solvents, lipids co-solvents, water-soluble co-solvents, and surfactants.

All the above features make the present formulations superior candidates for designing topically applied products for pharma and cosmetics.

Thus, it is another objective of the present invention to provide pharmaceutical compositions comprising therapeutically effective amounts of the formulations as above. The terms “effective amount” or” therapeutically effective amount” or “dose” imply an amount of active needed to provide a desired level of medicinal or non-medicinal effect. In some instances (clinical context) such effects are measurable by phenotypic parameters on other instances they are completely subjective (non-clinical context). For medicinal or therapeutic purposes, effective doses are usually derived from clinical trials and measured outcomes of clinical efficacy and safety.

In numerous embodiments the pharmaceutical compositions of the invention can be used in treating many common skin disorders or clinical conditions, and especially conditions involving cutaneous and mucosal inflammation such as psoriasis, eczema, acne, skin rashes, contact dermatitis, rosacea, hives, shingles, sun burns, and others.

This aspect can be further articulated in the form of methods for treating disorders or clinical conditions of the skin in respective subjects by topical administering to the subject the pharmaceutical compositions of the invention.

And further articulated in the form of uses of the pharmaceutical compositions of the invention for the manufacture of medicaments for treating disorders and clinical conditions of the skin.

One of the important objectives is to provide cosmetic compositions comprising effective amounts of the formulations of the invention.

Another objective is to provide cosmetic delivery systems comprising the formulations of the invention. The delivery systems can be further adapted for controlled or sustained release of the formulated cannabinoid actives and other ingredients onto the epidermal layer of the skin.

The cosmetic compositions and the delivery systems of the invention can be provided in a variety of forms and consistencies such as creams, lotions, gels, cosmetic masks, sprays, solid or liquid soaps or shampoos.

In numerous embodiments the present formulations can serve as a basis for cosmetic products in one or more of the following categories (recognized by the FDA):
01. Baby Products
02. Bath Preparations
03. Eye Makeup Preparations
04. Fragrance Preparations
05. Hair Preparations (non-coloring)
06. Hair Coloring Preparations
07. Makeup Preparations (not eye)
08. Manicuring Preparations
09. Oral Hygiene Products
10. Personal Cleanliness
11. Shaving Preparations
12. Skin Care Preparations (Creams, Lotions, Powders, and Sprays)
13. Suntan Preparations.

In numerous embodiments the present formulations can serve as a basis for cosmetic products for protecting the skin.

In numerous embodiments the skin protective property of the formulations is revealed in one or more of the following effects: moisturizing, soothing the skin and/or protecting the skin against photo- or oxidative stress, skin inflammation and/or skin aging.

In further embodiments the skin protective property of the formulations can comprise reducing the expression of one or more pro-inflammatory cytokine in the epidermal layer of the skin. The levels of the cytokines can be measured in quantified in various layers of the skin using known technologies such as ELISA, PCR, immunohistochemistry, and others.

In certain embodiments the skin protective property of the formulations can comprise reducing the expression of one or more interleukins IL-6, IL-8 and/or IL-1β in the epidermal layer of the skin.

The invention can be further articulated in terms of methods and uses for cosmetic purposes.

In numerous embodiments the cosmetic methods of the invention are meant to protect a subject's skin by topical administering or application of the present formulations. The formulations can be applied several times a day in specified intervals, and further, daily, weekly or monthly, depending on the intended use.

In numerous embodiments the cosmetic methods of the invention can provide one or more of the following effects: moisturizing, soothing the skin and/or protecting the skin against photo- or oxidative stress, skin inflammation and/or skin aging.

In further embodiments the cosmetic methods can provide a reduction of the expression of one or more pro-inflammatory cytokine in the epidermal layer of the skin.

In certain embodiments the cosmetic methods can provide a reduction of the expression of one or more interleukins IL-6, IL-8 and/or IL-1β in the epidermal layer of the skin.

Ultimately, the invention can be articulated in terms of use of any of the presently disclosed formulations in the manufacture of a cosmetic product for protecting the skin.

The term “about” in all its appearances in the text denotes up to a ±10% deviation from the specified values and/or ranges, more specifically, up to ±1%, ±2%, ±3%, ±4%, ±5%, ±6%, ±7%, ±8%, ±9% or ±10% deviation therefrom.

### EXAMPLES

Some embodiments of the invention will be now described by way of examples with reference to respective figures. Any method or material similar or equivalent to those described herein can be used in the practice or testing of the present invention.

### Materials and Methods

The materials used in the following examples are listed in Table 2.

TABLE 2
List of materials

| Materials | Manufacturer |
|---|---|
| Cannabidiol | Symrise AG |
| Cetearyl Alcohol (and) Cetearyl | Seppic |
| Glucoside | |
| Hydroxyethyl urea | AkzoNobel Surface Chemistry |
| 1,3-propanediol (Trimethylene glycol) | DuPont Tate & Lyle Bio |
| | Products |
| Sodium Hyaluronate | Givaudan Active Beauty |
| Phenoxyethanol (and) Caprylyl Glycol | Evonik Dr. Straetmans GmbH |
| Chlorphenesin | DKSH |
| Dimethicone | Xiameter (Dow corning) |
| Cyclopentasiloxane | Xiameter (Dow corning) |
| Glycerin (and) Glyceryl polyacrylate gel | BASF |
| Glyceryl Stearate SE | Faci |
| Cetearyl Alcohol | BASF |
| C12-15 Alkyl Benzoate | Saboderm |
| Cetearyl Ethylhexanoate | Saboderm |
| Theobroma Cacao (Cocoa) Seed Butter | Ziv chemical |
| Polysorbate 80 | Ziv chemical |
| Medium-chain Triglyceride (MCT) | Ziv Chemical |
| Sesame oil | H. Lamotte |
| Propyl Gallate | Sigma |
| Butylated hydroxy toluene (BHT) | Sigma |
| Ethanol | J. T. Baker |
| Methanol | J. T. Baker |
| Glycerin | Khen Shmuel Chemicals |
| Dimethyl sulfoxide (DMSO) | J. T. Baker |
| Acetonitrile | J. T. Baker |
| Ammonium Acetate | Fisher Scientific |
| Acetic acid | Bio-Lab |
| Disodium EDTA | Fagron |
| 1M Hydrochloride (HCl) | J. T. Baker |
| 1M Sodium hydroxide (NaOH) | J. T. Baker |
| Hydrogen peroxide (H₂O₂) 30% | Bio-Lab |

### I. CBD Formulation and Creams

The cosmetic formulations and creams of the invention were prepared by the following method:
1. Adding the oil phase (e.g., Crodamol ab, Crodamol cap, Dimethicone, Cyclopenta-siloxane, Cacao butter, GMS SE, Montanov 68, and Lanette O) to the aqueous phase (e.g., water, Disodium EDTA, Glycerin, Zemea, Hydrovance, Crystalhyal, Hispagel-200, Versatile pc, and Chlorphenesin) while stirring at 70° C. until the formation of a white homogenous cream;
2. Adding the cannabinoid (e.g., CBD, BHT, sesame oil, Tween 80) to the pre-heated cream (40° C.) and mixing at 1000 rpm, 30 min.

The main variables during the formulation process were reducing the concentration of ethanol and selecting for suitable antioxidants and oil solvents.

### II. Determination of CBD Content

The cosmetic formulations and creams of the invention were analyzed for CBD content by a specifically developed extraction method and HPLC.

### CBD Extraction

The formulations were subjected to various of methods of CBD extraction to achieve maximum efficiency by the extracted CBD vs. the initial CBD content (CBD assay %). The methods and results are listed in Table 3. Extraction Method #11 was more efficient and reproducible than other methods and used in the following studies.

TABLE 3
Different CBD extraction methods
CBD assay was calculated as triplicates (n = 3 ± STDEV)

| | | Cream | | CBD |
|---|---|---|---|---|
| | | weight | | assay |
| Method | Solvent | (mg) | Method steps | (%) |
| #1 | Methanol | 50 | 1 sonicator, 2 h shake | 94 ± .7 |
| #2 | | 100 | 30 min sonicator, 1 h | 93.2 ± 1.9 |
| #3 | | | 30 min sonicator, 2 h | 96.1 ± 1.4 |
| #4 | | | 1 h sonicator, 1 h shake | 96.5 ± .6 |
| #5 | | | 1 h sonicator, 2 h shake | 95.8 ± 2.2 |
| #6 | | 200 | 30 min sonicator, 1 h | 91.3 ± 3 |
| #7 | | | 30 min sonicator, 2 h | 88.7 ± 2.6 |
| #8 | | | 1 h sonicator, 1 h shake | 90.6 ± 1 |
| #9 | | | 1 h sonicator, 2 h shake | 88.8 ± 3.4 |
| #10 | | | 1 h sonicator, 4 h shake | 95.5 ± 0.9 |
| #11 | DMSO | 200 | 30 min sonicator, 1 h | 106.2 ± 2.5 |
| #12 | | | 30 min sonicator, 2 h | 101.2 ± 5 |

### Sample Preparation for HPLC

Samples were prepared for HPLC analysis by the following method:
1. Centrifugation at 4000 rpm for 5 min,
2. Filtration of 1.5 mL using 0.22 μm PTFE filters,
3. Dilution of 500 μL filtered sample in methanol to final concentration of 50 μg/mL CBD (10 ml volumetric flask using a glass pipette).
   HPLC analysis

HPLC analysis was performed under the following conditions:
1. Column—Luna Omega C18 3 μm, 150×4.6 mm
2. Guard—Security Guard TM Polar C18, 4×3.0 mm
3. Detector—220 nm
4. Flow rate—1 mL/min
5. Injection volume—10 μL
6. Column temperature—25° C.
7. Mobile phase—Gradient elution
8. Eluent A—Acetonitrile
9. Eluent B-10 mM Ammonium Acetate adjusted to pH 4.75 with Acetic Acid
10. Eluent C-Water

Gradient elution conditions are listed in Table 4. The same HPLC method can be applied to all primary cannabinoids (THC, THCA, CBD, CBDA, CBN, CBG, CBGA and CBC).

TABLE 4
Gradient mode

| Time (min) | Eluent A (%) | Eluent B (%) | Eluent C (%) |
|---|---|---|---|
| 0 | 66 | 34 | 0 |
| 0.5 | 66 | 34 | 0 |
| 1.5 | 82 | 18 | 0 |
| 6 | 82 | 18 | 0 |
| 8 | 85 | 15 | 0 |
| 11 | 85 | 15 | 0 |
| 12 | 85 | 0 | 15 |
| 14 | 100 | 0 | 0 |
| 35 | 100 | 0 | 0 |
| 37 | 66 | 0 | 34 |
| 38 | 66 | 34 | 0 |
| 45 | 66 | 34 | 0 |

### Samples Preparation for CBD Assay

Samples were prepared by diluting 500 μL cream in methanol to final concentration of 50 μg/mL CBD (10 mL volumetric flask and vortex for 10 sec). Assay percentage was calculated according to the following equation:
Asam—Area of peak of due to injected sample solution
Astd—Average peak area due to working standard solution
Csam—Concentration of the injected sample solution, μg/mL
Cstd—Concentration of the working standard solution, μg/mL.

### Samples Preparation for Force Degradation

Samples were prepared by the following method:
1. 200 mg 2% CBD cream vs. Blank (7 mL vials),
2. Subjected to Oxidation/Base/Acid/Heat/UV conditions (see below),
3. Mixing with 4 mL DMSO,
4. Sonication, 30 min,
5. Shaking, 1 h,
6. Centrifugation at 4000 rpm, 5 min,
7. Filtration by 0.22 μm PTFE,
8. HPLC sample preparation and HPLC protocol as above.

### Force Degradation Conditions

Samples were subjected to the following force degradation conditions and tested for CBD content:
Heat: 200 mg cream was heated at 100° C. (oil bath) for 6 h;
UV: 200 mg cream was exposed to the sun for 3 h;
Acidic conditions: 200 mg cream was treated with 150 μl 9M HCl (aq.) for 1 h and neutralized with 150 μl 9M NaOH;
Basic conditions: 200 mg cream was treated with 200 μl 1M NaOH for 2 h and neutralized with 200 μl 1M HCl;
Oxidation: 200 mg of cream was treated with 1 ml 30% H₂O₂ for 2 h; CBD assay % was calculated according to the untreated reference.

HPLC data on the specific force degradation conditions is shown in Tables 5-9, all data is summarized in Table 10.

TABLE 5
HPLC peak analysis in heat conditions

| Peak No. | Molecule | RT | Area mAU*min | RRT |
|---|---|---|---|---|
| 1 | | 4.243 | 0.1946 | 0.751 |
| 2 | | 4.439 | 0.0486 | 0.786 |
| 3 | | 5.387 | 0.1524 | 0.956 |
| 4 | CBD | 5.647 | 22.2512 | 1 |
| 5 | | 6.017 | 0.1189 | 1.065 |

TABLE 6
HPLC peak analysis in UV conditions

| Peak No. | Molecule | RT | Area mAU*min | RRT |
|---|---|---|---|---|
| 1 | CBD | 5.650 | 24.871 | 1 |

TABLE 7
HPLC peak analysis in oxidation conditions

| Peak No. | Molecule | RT | Area mAU*min | RRT |
|---|---|---|---|---|
| 1 | CBD | 5.657 | 20.058 | 1 |

TABLE 8
HPLC peak analysis in basic conditions

| Peak No. | Molecule | RT | Area mAU*min | RRT |
|---|---|---|---|---|
| 1 | CBD | 5.657 | 22.755 | 1 |

TABLE 9
HPLC peak analysis in acidic conditions

| Peak No. | Molecule | RT | Area mAU*min | RRT |
|---|---|---|---|---|
| 1 | | 4.437 | 0.0343 | 0.784 |
| 2 | CBD | 5.657 | 18.4206 | 1 |
| 3 | | 6.283 | 0.075 | 1.11 |

TABLE 10
Summary of force degradation data (CBD assay %)

| Condition | Heat | UV | Oxidation | Acid | Base |
|---|---|---|---|---|---|
| Time point | 6 h | 3 h | 2 h | 1 h | 1 h |
| CBD assay | 87.8% | 98.4% | 63.5% | 67.7% | 81.9% |

### III. Stability/Shelf-Life Studies

For stability studies, samples of the creams of the invention were stored in three different containers (5 g laminated tube, 10 mL white plastic jar and 20 g glass jar) at two different temperatures (25° C. and 40° C.) under the following conditions:
1. Long term conditions: 25° C.±2° C./60% RH±5% RH for 24 months.
2. Accelerated conditions: 40° C.±2° C./75% RH±5% RH for 6 months.

Samples were tested at specified time intervals (baseline and 1, 3, 6, 9, 12 and 24 months). The results are summarized in Table 11.

TABLE 11
Stability tests and specifications for scale-up

| | | | Sample amount | Time |
|---|---|---|---|---|
| Tests | | Specification | (g) | interval |
| 1 | Appearance | Color: White | | All |
| | | Form: Homogenous without granules | | |
| 2 | Foreign matter | Not found | 3 | All |
| 3 | CBD assay | 90-110% (0.9-1.1% w/w) | 0.6 | All |
| 4 | Identification | CBD retention time is identical to the standard | | All |
| 5 | pH | 5-7 | 1 | All |
| 6 | Microbiology | Total aerobic microbial count (cfu/g) - NMT 10² | 80 | T₀ |
| | | Total combined yeast count (cfu/g) - NMT 10¹ | | 40° C. 6 months |
| | | Absence of Staphylococcus aureus - NMT 1 g | | 25° C. 12 months |
| | | Absence of Pseudomonas aeruginosa: NMT 1 g | | |

### IV. Anti-Microbial Preservation Efficacy

For evaluation of antimicrobial preservation efficacy, the creams were subjected to a challenge test, as per the requirements of Ph. Eur. 5.1.3 (European Pharmacopoeia). Samples of the creams containing various preservatives at different concentrations (0.5% and 1%) were exposed to the relevant microorganisms (Staphylococcus aureus (ATCC 6538), Pseudomonas aeruginosa (ATCC 9027), Candida albicans (ATCC 10231), Aspergillus brasiliensis (ATCC 16404)) and tested at specific time intervals (baseline, 6 h, 24 h, 2 days, 7 days, 14 days and 28 days) vs. blank in the same conditions.

### V. Dermal Bio-Distribution (Ex-Vivo)

Skin penetration was evaluated using the static diffusion method, Franz cells (Perme Gear; Hellertown, PA, USA). Human skin samples were cleaned from the hypodermis tissue and fat and measured (thickness of about 0.7-0.8 mm). Formulations containing 100 μg CBD were added to the donor compartment. At the end of the study, the samples were processed as follows for the subsequent HPLC analysis:
1. Receptor samples were collected (glass tubes), dried until complete dryness (water bath at 50° C., air flow 10 KPA), reconstituted in acetonitrile;
2. Donor samples were collected by washing with 0.5 ml distilled water and 0.5 ml (×2) acetonitrile, the skin sample was rubbed with a cotton-swab, placed with the extract (2 ml Eppendorf), subjected to centrifugation at 14,000 rpm, 10 min, the supernatant was collected and diluted with acetonitrile;
3. Residual skin (the area surrounding skin that was in contact with donor) was cut in small pieces, transferred to tubes (5 ml plastic conical centrifuge tubes), mixed with 3 ml acetonitrile and homogenized (Polytron PT 10-35 GT) at 20,000 rpm, 5 min on ice, centrifuged at 4400 rpm, 10 min, the supernatant was collected (2 ml Eppendorf), diluted with acetonitrile and centrifuged at 14,000 rpm, 10 min;
4. Stratum corneum (SC) was separated from the portion which was in contact with the donor using the tape-stripping technique (by an adhesive tape ×5), the strips were placed in 20 ml scintillation vials containing 2 ml acetonitrile and vortexed, 30 min, the solution was collected, centrifuged at 14,000 rpm, 10 min;
5. Epidermal layer was scraped from the dermis with a scalpel and transferred to a tube (2 ml Eppendorf), mixed with 1 ml acetonitrile and homogenized, centrifuged at 14,000 rpm, 10 min, followed by same steps as for the residual skin fraction;
6. Dermis was cut into small pieces and transferred to a tube (15 ml plastic conical centrifuge tube) mixed with 3 ml of acetonitrile, followed by same steps as for the residual skin fraction.

### VI. Anti-Inflammatory Activity Using LPS Model (Ex-Vivo)

The skin samples obtained as above were maintained at 37° C. in 5% CO₂ in serum-free DMEM containing 2 mM L-glutamine, 100 IU/mL penicillin and 100 μg/mL streptomycin. Samples were treated with 5-10 μg/ml Lipopolysaccharide (LPS) added to the growth medium to induce inflammation. The tested preparations were applied topically to the skin samples after LPS activation (48 h), including the CBD formulation #34 or #27 (15 μg/cm²), vehicle control (MCT) and clobetasol propionate as a steroid reference (2.5 μg/cm²). The levels of pro-inflammatory cytokines (IL-8, IL-6 and IL-1β) were determined in the medium by ELISA in the treated in the untreated groups after 24 h. The experiment was performed in ×6 replicates.

An analogous experiment testing the effect of the CBD formulations with various types of oil (MCT and sesame oil) used dexamethasone as a steroid reference and tested for pro-inflammatory cytokines IL-6 and IL-1β.

### Example 1 Development of CBD Containing Creams of the Invention

Several examples of the CBD containing formulations and creams of the invention are provided in Table 12. The main tested parameters included the following:
1. Reduction of ethanol: Ethanol is generally considered a co-solvent of CBD. The concentration of ethanol was reduced from initial to final preparations, starting from 3.6% and 0.9% to no ethanol in the final preparations, where CBD was dissolved directly in sesame oil (see for example CBD #34 in Table 13 below).
2. Selection of an antioxidant: The selected antioxidants were BHT and propyl gallate, in various combinations and proportions. It was found that propyl gallate affects the color of the preparation, but not BHT. Due to this property, the final preparations contained 0.05% BHT (see Table 13, CBD #34).
3. Selection of an oil: Oil is a known solvent for CBD. Various oils were tested as optional solvents in the cosmetic creams of the invention. Both, medium-chain triglycerides (MCT) and sesame oil were found suitable to provide a complete dissolution of CBD (2%). Both MCT and sesame oil have antioxidants properties (see Table 13, CBD #34).

TABLE 12
Various CBD containing creams of the invention

| | CBD22 | CBD 23 | CBD 24 | CBD 25 | CBD 27 |
|---|---|---|---|---|---|
| Ingredient | % w/w | % w/w | % w/w | % w/w | % w/w |
| Water | 54.3 | 53.23 | 53.23 | 53.23 | 54.3 |
| Disodium EDTA | 0.05 | 0.05 | 0.05 | 0.05 | 0.05 |
| Glycerin | 3.28 | 3.43 | 3.43 | 3.43 | 3.28 |
| Zemea | 3.28 | 3.43 | 3.43 | 3.43 | 3.28 |
| Hydrovance | 3.28 | 3.43 | 3.43 | 3.43 | 3.28 |
| Crystalhyal | 0.54 | 0.7 | 0.7 | 0.7 | 0.54 |
| Hispagel-200 | 2.18 | 2.33 | 2.33 | 2.33 | 2.18 |
| Versatil PC | 1.09 | 1.10 | 1.10 | 1.10 | 1.09 |
| Chlorphenesin | 0.2 | 0.20 | 0.20 | 0.20 | — |
| Crodamol AB | 3.28 | 3.42 | 3.42 | 3.42 | 3.28 |
| Crodamol CAP | 1.64 | 1.78 | 1.78 | 1.78 | 1.64 |
| 350 | 0.54 | 0.69 | 0.69 | 0.69 | 0.54 |
| 1202 | 4.37 | 4.52 | 4.52 | 4.52 | 4.37 |
| Cacao butter | 3.73 | 2.94 | 2.94 | 2.94 | 2.73 |
| GMS SE | 3.28 | 3.42 | 3.42 | 3.42 | 3.28 |
| Montanov 68 | 5.47 | 5.61 | 5.61 | 5.61 | 5.47 |
| Lanette O | 4.92 | 5.06 | 5.06 | 5.06 | 4.92 |
| Ethanol | 3.3 | 3.3 | 3.3 | 3.3 | 0.9 |
| MCT | 2 | 2 | 2 | 2 | 2.26 |
| Tween 80 | 0.3 | 0.30 | 0.30 | 0.30 | 0.34 |
| CBD | 2.0 | 2.00 | 2.00 | 2.00 | 2.00 |
| BHT | | — | 0.05 | 0.05 | 0.05 |
| Propyl gallate | | 0.1 | | 0.1 | 0.10 |
| Appearance at 6 | No | Brown | No | Brown | Beige |
| weeks (T0 = | change | | change | | |
| white) | | | | | |
| CBD content (%) | No | No | No | No | No |
| | change | change | change | change | change |

An example of advantageous CBD formulation of the invention (CBD #34) by the parameters of a complete CBD dissolution and prolonged stability is demonstrated in Table 13.

TABLE 13
Ingredients in the example formulation CBD#34

| Ingredients | INCI Name | % | Function |
|---|---|---|---|
| Water | | 53.22 | |
| Disodium EDTA | Edetate Disodium | 0.05 | Chelating agent |
| Glycerin | Glycerin | 3.4 | Humectant |
| Zemea | 1,3-propanediol | 3.4 | Humectant, emollient, boosts efficacy of |
| | (Trimethylene glycol) | | preservatives |
| Hydrovance | Hydroxyethyl urea | 3.4 | Hydrate skin and help maintain a normal |
| | | | moisture balance in skin's uppermost layers |
| Crystalhyal | Sodium Hyaluronate | 0.7 | High molecular weight hyaluronic acid: |
| | | | moisturize and protect |
| Hispagel-200 | Glycerin (and) Glyceryl | 2.3 | Moisturize |
| | polyacrylate gel | | |
| Verstatil PC | Phenoxyethanol (and) | 1.1 | Preservative blend |
| | Caprylyl Glycol | | |
| Chlorphenesin | Chlorphenesin | 0.2 | Preservative |
| Saboderm AB | C12-15 Alkyl Benzoate | 3.4 | Solvent and solubilizer and preservative |
| Saboderm CSO | Cetearyl | 1.8 | Emollient |
| | Ethylhexanoate | | |
| Xiameter PMX- | Dimethicone | 0.7 | Soft feel and subtle skin lubricity, |
| 200 Sil Fluid | | | Nongreasy feel |
| 350cs | | | |
| SF- 1202 | Cyclopentasiloxane | 4.5 | Offers greater flexibility in formulating |
| | | | cosmetic product; dry non-greasy feel |
| Cocoa butter | Theobroma Cacao | 2.9 | Emollient, rich source of antioxidant |
| | (Cocoa) Seed Butter | | |
| GMS SE | Glyceryl Stearate SE | 3.4 | Self-emulsifying base |
| Montanov 68 | Cetearyl Alcohol (and) | 5.6 | O/W emulsifiers |
| | Cetearyl Glucoside | | |
| Lanette O | Cetearyl Alcohol | 5.1 | Viscosity regulations in cosmetic oil-in- |
| | | | water emulsions |
| CBD | Cannabidiol | 2.0 | Active |
| Tween 80 | Polysorbate 80 | 0.3 | CBD solvent |
| Sesame oil | Sesamum Indicum | 2.3 | CBD solvent |
| | (Sesame) Seed Oil | | |
| BHT | Butylated | 0.05 | Antioxidant |
| | hydroxytoluene | | |

### Example 2 Stability Studies

CBD #34 was used as an example product in the stability/shelf-life studies under the following storage conditions:
1. different containers, a laminated tube (5 g), a white plastic jar (10 mL) and a glass jar (20 g); and.
2. different storage temperatures, 25° C. and 40° C.

Samples were analyzed at the specified time intervals (baseline, 1, 3, 6, 9, and 12 months) for the general appearance of color and texture (white and ungranulated), the presence of foreign material, CBD content (assay % and retention time), pH and microbial growth (total aerobic microbial count, total combined yeast/mold count, absence of Staphylococcus aureus and Pseudomonas aeruginosa).

Surprisingly, CBD #34 was found stable by all the above parameters. In the laminated tube, the general appearance of color and texture was preserved at 25° C. for a period of at least 6 months and turned yellow only after 9 months in these conditions. The change of color was more rapid (after 3 months) at 40° C. in same container. In the glass and plastic jars, the formulations changed color more rapidly (after 1 to 3 months), at both temperatures. In the laminated tube, the CBD assay remained in the specification range (90-110%) at 25° C. for at least 12 months. CBD was confirmed at all time points. Foreign matter was not detected in any conditions. pH remained in the specification range in all conditions. Microbial assays detected no growth after 6 and 12 months.

These stability data support the use of CBD #34 as a basis for a commercial product. They further provide concrete guidelines on the specific storage conditions ensuring the preservation of the cosmetic product and the availability of CBD.

### Example 3 Antimicrobial Preservation—a Challenge Test

The formulation CBD #34 with phenoxyethanol 0.5% (w/w) was further tested in a challenge test for antimicrobial preservation, the test was performed by a certified lab. The results are shown Table 14. Overall, it was found that the product was preserved in a satisfactory manner that meets the requirements of Ph. Eur. 5.1.3.

TABLE 14
Efficacy of antimicrobial preservation

| | Contamination (CFU/g) | | | | | | |
|---|---|---|---|---|---|---|---|
| Test microorganism | T0 | 6 h | 24 h | 2 days | 7 days | 14 days | 28 days |
| Staphylococcus aureus | 2.2*10⁵ | N/A | N/A | 1.1*10 | <10 | N/A | <10 |
| Pseudomonas aeruginosa | 2.4*10⁵ | N/A | N/A | <10 | <10 | N/A | <10 |
| Candida albicans | 1.9*10⁵ | N/A | N/A | N/A | N/A | <10 | <10 |
| Aspergillus brasiliensis | 2.2*10⁵ | N/A | N/A | N/A | N/A | 1.6*10² | <10 |
| Uninoculated Control | <10 | N/A | N/A | <10 | <10 | <10 | <10 |

### Example 4 Dermal Bio-Distribution (Ex-Vivo)

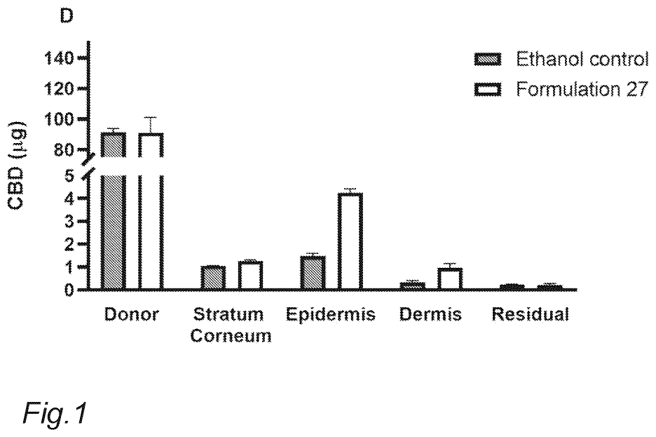

The fresh formulation CBD #27 was tested on various layers of the skin (the stratum corneum, epidermis, dermis, residual layers) compared to the CBD-ethanol reference, using the static diffusion method (Franz cells) in donor and receptor compartments. CBD #27 was applied topically in the amount of 5 mg (˜100 μg CBD) and CBD-ethanol in the amount of 10 μl (˜100 μg CBD) (see Material and Methods). The weights of the tested skin layers are shown in Table 15. The results are summarized in Table 16 and FIG. 1. For all cases, CBD was undetected in the receptor compartment (limit of detection, LOD=10 ng/ml).

TABLE 15
Weights of different layers of the skin

| Formulations | Epidermis (mg) | Dermis (mg) | Residual (mg) |
|---|---|---|---|
| CBD#27 (100 μg) | 12.42 ± 1.69 | 95.23 ± 7.46 | 97.08 ± 7.21 |
| CBD-ethanol (100 μg) | 12.18 ± 1.32 | 103.11 ± 5.29 | 93.22 ± 7.69 |

TABLE 16
Amount of CBD in different layers of skin

| | Donor | Stratum Corneum | Epidermis | Dermis | Residual Layer |
|---|---|---|---|---|---|
| Formulations | (μg) | (μg) | (μg) | (μg) | (μg) |
| FOR 27 (100 μg) | 91.24 ± 10.11 | 1.27 ± 0.05 | 4.25 ± 0.18 | 0.97 ± 0.19 | 0.22 ± 0.06 |
| CBD-ethanol (100 μg) | 91.42 ± 2.60 | 1.06 ± 0.01 | 1.47 ± 0.14 | 0.33 ± 0.08 | 0.23 ± 0.03 |

CBD #27 uses MCT (2.26% w/w) as an oil solvent and a low concentration of ethanol (0.9% w/w) (see Table 12). The results show that CBD #27 is more efficient than CBD-ethanol by 3-fold in enhancing the penetration of CBD, predominantly into the epidermal layer. Ethanol is known to enhance the skin penetration of lipophilic drugs, but its chronic use was cautioned against because of safety concerns (Lachenmeier DW 2008. “Safety evaluation of topical applications of ethanol on the skin and inside the oral cavity”. J Occup Med Toxicol 3:26-41). No CBD was observed in the receptor compartment, suggesting a localized and restricted CBD penetration.

### Example 5 Anti-Inflammatory Activity in the LPS Model (Ex-Vivo)

CBD #27 was further tested for anti-inflammatory activity in the LPS model (artificially induced inflammation) and measuring the levels of pro-inflammatory cytokines (IL-6, IL-8 and IL-1β), while comparing skin samples treated with CBD #27 (2% CBD applied topically 15 μg/cm²), with a steroid control (clobetasole propionate ointment 2.5 μg/cm²), with a vehicle (MCT) and untreated samples (see Material and Methods). The experiment was performed in ×6 replicates.

The amounts of CBD in the different skin layers (1 g), comparing CBD #27 (100 μg) and CBD-ethanol (100 μg), are shown in Table 17, thus reproducing the previous data on the enhanced penetration of the formulations of the invention to the epidermis by 3-fold relative to the CBD-ethanol control (see Table 16).

TABLE 17
Amount of CBD in the different layers of the skin

| | Epidermis | Dermis | Residual |
|---|---|---|---|
| Formulations | (μg/gm) | (μg/gm) | (μg/gm) |
| CBD#27 (100 μg) | 328.67 ± 11.59 | 9.52 ± 1.45 | 2.71 ± 0.36 |
| CBD-ethanol (100 μg) | 112.38 ± 6.25 | 3.39 ± 0.77 | 2.24 ± 0.28 |

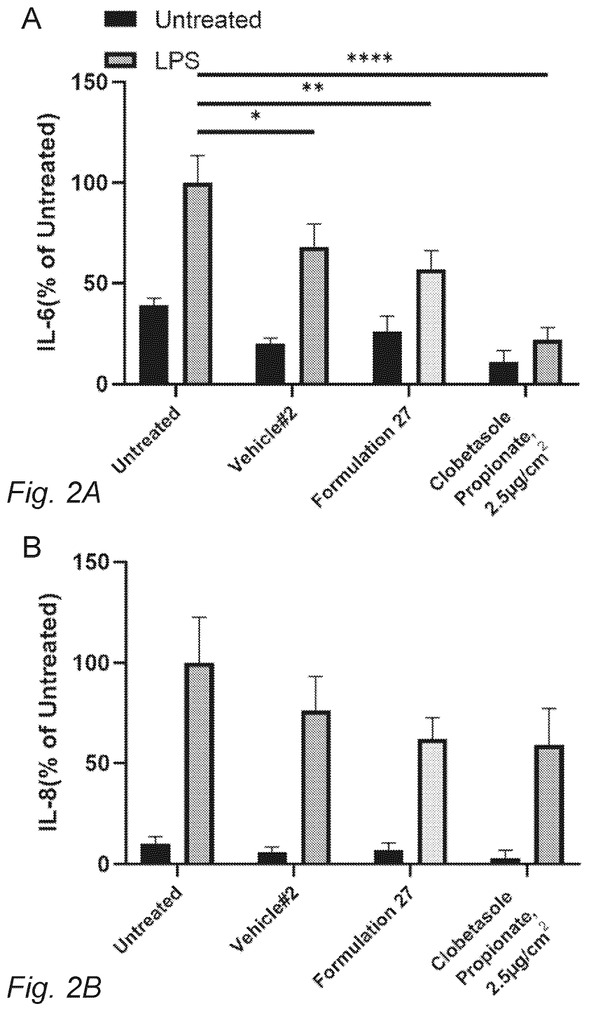

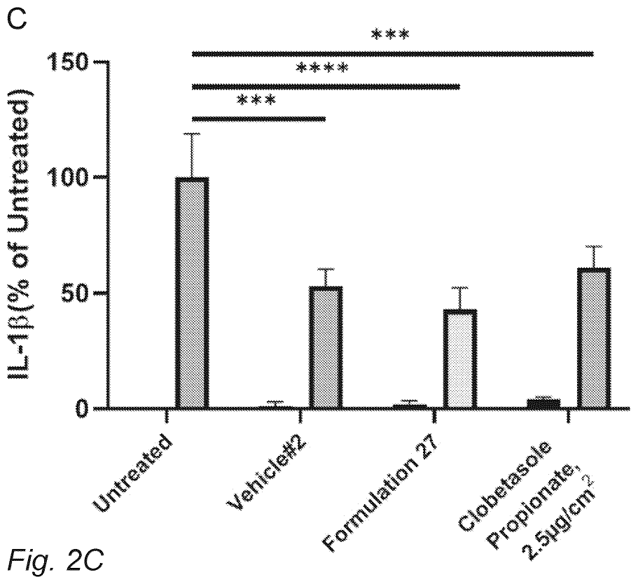

The anti-inflammatory effect of CBD #27 is demonstrated in FIGS. 2A-2C. CBD #27 significantly reduced the secretion of all tested pro-inflammatory cytokines, IL-6 (FIG. 2A), IL-8 (FIG. 2B) and IL-1β (FIG. 2C), with the most significant reduction in IL-1β which exceeded the steroid reference and in IL-8 which was similar thereto.

It was assumed that the observed anti-inflammatory effect of CBD #27 can be a combination of effects of CBD and additional components, such as excipients and solvents. Evidences of such additive or synergistic effect can be seen in the cytokine profile of Vehicle #2 (MCT), showing a partial reduction of the respective pro-inflammatory cytokines compared to CBD #27. It has been known that MCT (a mid-chain triglyceride with no double bonds) has a lower tendency to oxidation than other vegetable oil triglycerides containing esters of polyunsaturated fatty acids. According to this experiment, MCT further has mild anti-inflammatory properties contributing to additive and synergistic effects of CBD formulated therewith.

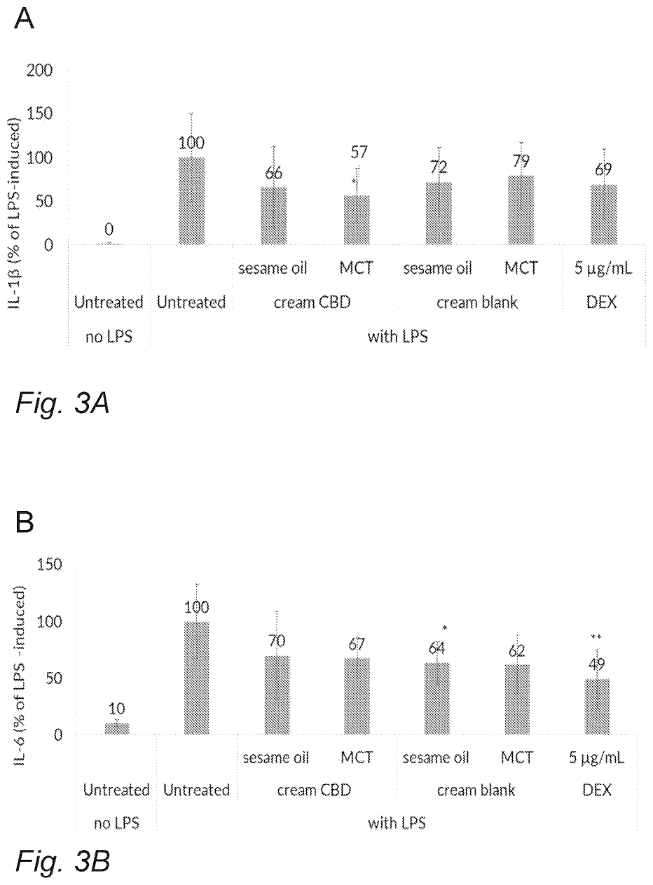

The effect of oils was further tested in analogous experiment using the CBD formulations of the invention with different oils (MCT and sesame oil) and dexamethasone as a steroid reference and measuring the levels of IL-6 and IL-1β. The results are shown in FIGS. 3A-3B. Both, sesame oil and MCT had anti-inflammatory effect on the secretion of IL-6 and IL-1β. In the CBD formulations of the invention, however, the effect of the oils was more significant, especially on IL-1β where it exceeded the effect of the steroid reference. This phenomenon should be further studied in larger experiments.

Overall, these studies demonstrate the capability of the present technology to provide a superior line of cannabinoid-based products for cosmetic applications, with an improved composition of actives affording a wide range of cosmetic benefits such as moisturizing, soothing, protective, antioxidant, anti-inflammatory and anti-aging effects, and further, an improved performance in terms of stability, epidermal delivery and cosmetic efficacy overall.

1-40. (canceled)

41. A topical water-based formulation comprising a cannabinoid material, ethylenediaminetetraacetic acid (EDTA) and an antioxidant comprising one or more of: butylated hydroxytoluene (BHT), butylated hydroxyanisole (BHA) and tert-butyl hydroquinone (TBHQ), the formulation being essentially devoid of an alcohol.

42. The topical formulation of claim 41, wherein the antioxidant further comprises at least one antioxidant oil, being a natural or a synthetic medium chain triglyceride (MCT) or a vegetable oil, or any combination thereof.

43. The topical formulation of claim 41, wherein the alcohol is present at a concentration in the range between about 0.001 to about 1% of the total weight (w/w) or is absent.

44. The topical formulation of claim 41, the formulation being stable at room temperature for at least 6 months.

45. The topical formulation of claim 41, wherein the cannabinoid material is characterized as having a preferential distribution to the epidermal layer of the skin compared to the other layers of the skin.

46. The topical formulation of claim 41, wherein the cannabinoid material is characterized as having a preferential distribution to the epidermal layer of the skin compared to the distribution of the respective cannabinoid control in the epidermal layer.

47. A cosmetic composition comprising an effective amount of a topical formulation of claim 41.

48. A cosmetic delivery system comprising a topical formulation of claim 41.

49. A cream, a lotion, a gel, a cosmetic mask, a spray, a solid or a liquid soap or a shampoo comprising the cosmetic composition of claim 47.

50. A cream, a lotion, a gel, a cosmetic mask, a spray, a solid or a liquid soap or a shampoo comprising the cosmetic delivery system of claim 48.

51. A pharmaceutical composition comprising a therapeutically effective amount of a topical formulation of claim 41.

52. A cosmetic composition for protecting the skin, the cosmetic composition comprises a topical formulation of claim 41.

53. The cosmetic composition for use according to claim 52, said protecting the skin having at least one of: a moisturizing, a soothing, an anti-photo- and/or an anti-oxidative affect and/or an anti-inflammatory or an anti-aging effect on the skin.

54. The cosmetic composition for use according to claim 53, said protecting the skin further comprising reducing the expression of at least one pro-inflammatory cytokine in the epidermal layer of the skin.

55. A cosmetic method for protecting a subject's skin, the method comprises topical administering of the cosmetic composition of claim 47.

56. The cosmetic method of claim 55, said protecting a subject's skin having at least one of: a moisturizing, a soothing, an anti-photo- and/or an anti-oxidative affect, and/or an anti-inflammatory or an anti-aging effect on the subject's skin.

57. The cosmetic method of claim 56, said protecting the skin further comprising reducing the expression of at least one pro-inflammatory cytokine in the epidermal layer of the skin.

58. The cosmetic method of claim 57, said reducing the expression of at least one pro-inflammatory cytokine in the epidermal layer of the skin comprises reducing the expression of at least one of: an interleukin IL-6, IL-8 and/or IL-1β in the epidermal layer of the skin.

59. The pharmaceutical composition of claim 51 for use in treating a disorder or a clinical condition of the skin.

60. A method for treating a disorder or a clinical condition of the skin in a subject in need thereof, the method comprises topical administering to the subject the pharmaceutical composition of claim 51.
